(19) United States
(12) Patent Application Publication
(10) Pub. No.: US 2026/0306308 A1
(43) Pub. Date: Oct. 8, 2026

(54) Swim Diaper Garment Device

(71) Applicants: Misty Spangler, Wisconsin Rapid, WI (US); Douglas Spangler, Wisconsin Rapid, WI (US)

(72) Inventors: Misty Spangler, Wisconsin Rapid, WI (US); Douglas Spangler, Wisconsin Rapid, WI (US)

(21) Appl. No.: 19/259,025

(22) Filed: Jul. 3, 2025

Related U.S. Application Data
(60) Provisional application No. 63/784,531, filed on Apr. 7, 2025.

(51) Int. Cl. A61F 13/49

(52) U.S. Cl. CPC A61F 13/49011; A61F 2013/49087; A61F 2013/49098

(57) ABSTRACT

A swim diaper garment device is provided. The device is comprised of a garment body that resembles conventional swimwear while incorporating waste management features suitable for aquatic environments. The device comprises a waist opening and leg openings bordered with elastic bands that incorporate dual-tension elastomeric filaments to ensure secure sealing and comfort. Internally, an absorbent layer comprised of hydrophobic nonwoven substrates and superabsorbent polymer elements is disposed to capture bodily fluids while resisting water saturation. Adjacent to the absorbent layer, a containment barrier layer formed from waterproof, stretchable materials provides leakage protection and movement adaptability. The exterior shell layer comprises swimwear-grade textiles with aesthetic treatments to mimic conventional swimwear and ensure social discretion. A saturation indicator strip may be included to signal when the device has reached absorbent capacity.

### CROSS-REFERENCE TO RELATED APPLICATION

The present application claims priority to, and the benefit of, U.S. Provisional Application No. 63/784,531, which was filed on Apr. 7, 2025, and is incorporated herein by reference in its entirety.

### FIELD OF THE INVENTION

The present invention relates generally to the field of diapers. More specifically, the present invention relates to a garment that resembles conventional swimwear while incorporating waste management features suitable for aquatic environments. Accordingly, the present disclosure makes specific reference thereto. Nonetheless, it is to be appreciated that aspects of the present invention are also equally applicable to other like applications, devices, and methods of manufacture.

### BACKGROUND

Incontinence presents a significant challenge for individuals who wish to engage in recreational swimming or aquatic therapy. People who suffer from urinary or fecal incontinence may be deterred from participating in water-based activities due to the risk of leakage and the associated social and hygienic consequences. In swimming environments, an incontinence-related accident can cause embarrassment for the individual and potential contamination of shared water, posing health and safety risks for other users. Conventional absorbent products, such as standard diapers, are not suitable for use in water, as their materials tend to absorb pool water, swell excessively, and lose structural integrity. This expansion can create discomfort, limit mobility, and reduce the effectiveness of the diaper in containing waste. Furthermore, traditional swimwear lacks the functional components necessary to manage incontinence, offering no absorbency or containment capabilities. The inability to discreetly and effectively manage bodily waste while swimming excludes many individuals from an activity that can otherwise offer physical, mental, and social benefits.

Therefore, there exists a long-felt need in the art for a swim diaper garment device that prevents the escape of waste while submerged in water. There also exists a long-felt need in the art for a swim diaper garment device that does not swell or lose function when exposed to aquatic environments. Moreover, there exists a long-felt need in the art for a swim diaper garment device that enables hygienic removal without contaminating outer garments or the surrounding area.

The subject matter disclosed and claimed herein, in one embodiment thereof, comprises a swim diaper garment device. The device is comprised of a garment body that resembles conventional swimwear while incorporating waste management features suitable for aquatic environments. The device comprises a waist opening and leg openings bordered with elastic bands that incorporate dual-tension elastomeric filaments to ensure secure sealing and comfort. Internally, an absorbent layer comprised of hydrophobic nonwoven substrates and superabsorbent polymer elements is disposed to capture bodily fluids while resisting water saturation. Adjacent to the absorbent layer, a containment barrier layer formed from waterproof, stretchable materials provides leakage protection and movement adaptability. The exterior shell layer comprises swimwear-grade textiles with aesthetic treatments to mimic conventional swimwear and ensure social discretion. A saturation indicator strip may be included to signal when the device has reached absorbent capacity. Tear-apart side seams positioned on the lateral flanks of the garment are configured to rupture under manual force, allowing for clean and sanitary removal after use.

In this manner, the swim diaper garment device of the present invention accomplishes all the forgoing objectives and provides a wearable device that enables incontinent individuals to participate in aquatic activities without the risk of embarrassment or environmental contamination. The integration of non-swelling absorbent materials and a watertight containment barrier provides reliable waste management under submerged conditions. Additionally, the incorporation of tear-apart side seams facilitates easy and hygienic removal, addressing practical concerns related to post-use cleanup. By offering discretion, functionality, and comfort, the device fulfills long-standing unmet needs in the field of incontinence products suitable for swimming environments.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a swim diaper garment device. The device is comprised of a body shaped in standard swimwear silhouettes, such as briefs or trunks, to support discretion, ergonomic fit, and swimming performance. The body is designed as a slip-on garment without mechanical fasteners, adhesives, or closures, simplifying donning and removal while reducing failure risks and pressure points.

The body includes leg and waist openings, each bordered with elastic bands. The leg openings are anatomically shaped to reduce pressure and support freedom of movement. The elastic bands are comprised of multi-strand elastomeric filaments with dual-tension properties, providing secure sealing and user comfort, while maintaining stretchability to support slip-on functionality. The body may also be comprised of tear-apart side seams that are positioned along the lateral sides of the body and that are configured to rupture under manual force for hygienic removal.

An absorbent layer is disposed within the body to manage bodily fluids while resisting water saturation during aquatic use. A containment barrier layer is positioned adjacent to or enclosing the absorbent layer and is comprised of waterproof, flexible materials. The barrier layer is configured for multi-directional deformation to support body movement and includes a hydrophobic finish to enhance water resistance. The exterior of the body is covered with a shell layer comprised of swimwear-grade textiles. The shell layer maintains the visual and tactile characteristics of conventional swimwear and may include aesthetic treatments to obscure internal components and promote discretion.

A saturation indicator strip may be included to provide a visible color change upon reaching a threshold level of moisture or waste detection. The indicator strip is comprised of a colorimetric material and may be embedded within or affixed to the absorbent or barrier layer to provide a non-verbal cue for disposal.

A method of use is also provided, comprised of donning the device by pulling the body upward over the legs and hips until the waist and leg openings align anatomically. The user may engage in aquatic activities while the absorbent and barrier layers manage waste and prevent saturation. The saturation indicator is monitored to determine when replacement is needed. The device is then removed by tearing the side seams and is discarded according to sanitary procedures.

Accordingly, the swim diaper garment device of the present invention is particularly advantageous as it provides a wearable device that enables incontinent individuals to participate in aquatic activities without the risk of embarrassment or environmental contamination. The integration of non-swelling absorbent materials and a watertight containment barrier provides reliable waste management under submerged conditions. Additionally, the incorporation of tear-apart side seams facilitates easy and hygienic removal, addressing practical concerns related to post-use cleanup. In this manner, the swim diaper garment device provides a solution allowing incontinent individuals to swim.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of a swim diaper garment device of the present invention in accordance with the disclosed architecture;

FIG. 2 illustrates a side cross-sectional view of a body construction of one potential embodiment of a swim diaper garment device of the present invention in accordance with the disclosed architecture; and

FIG. 3 illustrates a flowchart of a method of using one potential embodiment of a swim diaper garment device of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for a swim diaper garment device that prevents the escape of waste while submerged in water. There also exists a long-felt need in the art for a swim diaper garment device that does not swell or lose function when exposed to aquatic environments. Moreover, there exists a long-felt need in the art for a swim diaper garment device that enables hygienic removal without contaminating outer garments or the surrounding area.

The present invention, in one exemplary embodiment, is comprised of a swim diaper garment device. The device includes a body formed in standard swimwear silhouettes, including but not limited to briefs or trunks, in order to promote discretion, ergonomic fit, and effective performance during swimming. Constructed as a slip-on garment, the body eliminates the need for mechanical fasteners, adhesives, or closures, thereby simplifying the process of donning and removal while reducing the likelihood of mechanical failure and pressure-related discomfort.

The body further has leg and waist openings, with each opening bordered by elastic bands. The leg openings are contoured anatomically to reduce localized pressure and enhance freedom of movement. The elastic bands are comprised of multi-strand elastomeric filaments exhibiting dual-tension properties, where outer strands ensure sealing effectiveness and inner strands preserve wearer comfort. The elastic configuration also enhances slip-on functionality. Additionally, the body may include tear-apart side seams located along the lateral flanks, which are engineered to rupture under moderate manual force to facilitate hygienic removal.

An absorbent layer is positioned within the body for the containment of bodily fluids, designed to resist saturation during aquatic use. Adjacent to or enclosing this layer is a containment barrier layer comprised of flexible, waterproof materials. The barrier layer is capable of multidirectional deformation to accommodate body movement and is finished with a hydrophobic coating to improve water resistance. The outer surface of the body is covered by a shell layer comprised of swimwear-grade textiles, maintaining a visual and tactile similarity to conventional swimwear. This shell layer may include visual enhancements to obscure internal components and promote discretion. A saturation indicator strip may be incorporated to visually signal when the device has reached moisture or waste capacity. This strip is comprised of colorimetric material and may be embedded in or attached to the absorbent or barrier layer and provides a non-verbal indication that disposal is necessary.

A method of use is also disclosed. The device is donned by drawing the body upward over the legs and hips until the waist and leg openings are aligned with the wearer's anatomy. The user may then engage in aquatic activities while the absorbent and barrier layers manage excreted waste and prevent water ingress. The saturation indicator is observed for signs of capacity threshold, at which point the device is removed by manually tearing the side seams and discarded in accordance with proper sanitary procedures.

As a result, the device provides a practical solution for enabling incontinent individuals to participate in aquatic activities without risk of embarrassment or environmental contamination. The combination of non-swelling absorbent materials and a watertight containment barrier ensures effective waste management in submerged conditions. Furthermore, tear-apart side seams enhance hygienic handling, addressing key post-use concerns and supporting user dignity.

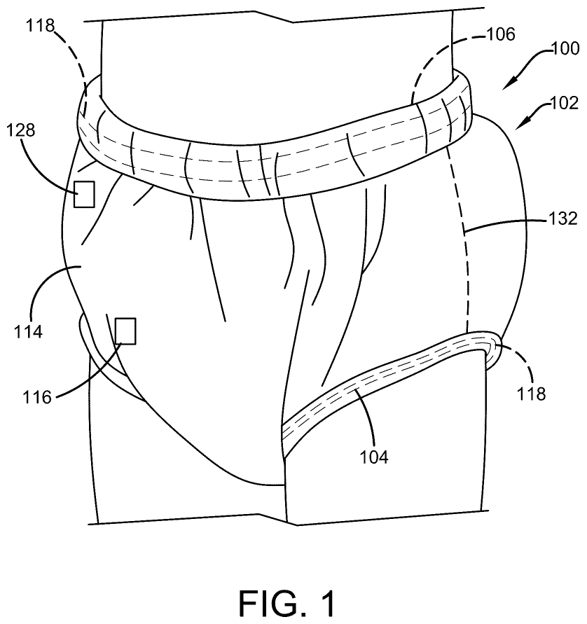

Referring initially to the drawings, FIG. 1 illustrates a perspective view of one potential embodiment of a swim diaper garment device 100 of the present invention in accordance with the disclosed architecture. The device 100 is a combined swimwear and absorbent garment intended for use in aquatic environments. The device 100 may provide an outward appearance consistent with conventional swimwear while incorporating internal features for the containment of bodily waste. The device 100 may be configured for single-use and designed for disposal following wear.

The device 100 may be comprised of a body 102. The body 102 may be shaped in accordance with standard swimwear silhouettes, including but not limited to briefs, trunks, boxer briefs, swim shorts, etc. The shape of the body 102 may be selected to ensure social discretion, ergonomic conformity, and performance suitable for swimming, including minimized drag and freedom of movement. The device 100 is preferably a slip-on garment, wherein the body 102 may be constructed as a continuous, seamless ring or partially seamed structure that can be pulled upward over the legs and hips without the need for mechanical fasteners, adhesives, or closures. The slip-on design may simplify donning and removal of the device 100, reduce the likelihood of mechanical failure during active use, and eliminate pressure points.

The body 102 may include leg openings 104 and a waist opening 106, as seen in FIG. 1. The leg openings 104 may be shaped in an anatomical arc to follow the natural curvature of the user's thighs, which may reduce localized pressure, minimize restriction during motion, and enhance comfort during both swimming and general wear. The waist opening 106 may encircle the torso at or near the pelvic crest to provide a secure fit while minimizing upward or downward migration during movement. Both the leg openings 104 and the waist opening 106 may be comprised of elastic bands 118. The elastic bands 118 may incorporate multi-strand elastomeric filaments exhibiting dual-tension behavior, wherein the outermost filaments may be configured to exert greater tension for enhanced sealing against fluid leakage, while the inner filaments may be engineered for lower tension to maintain user comfort. The elasticity of bands 118 may also support the slip-on functionality by providing sufficient stretchability for insertion over the feet and hips, followed by contraction to form a secure seal that conforms to the user's anatomy.

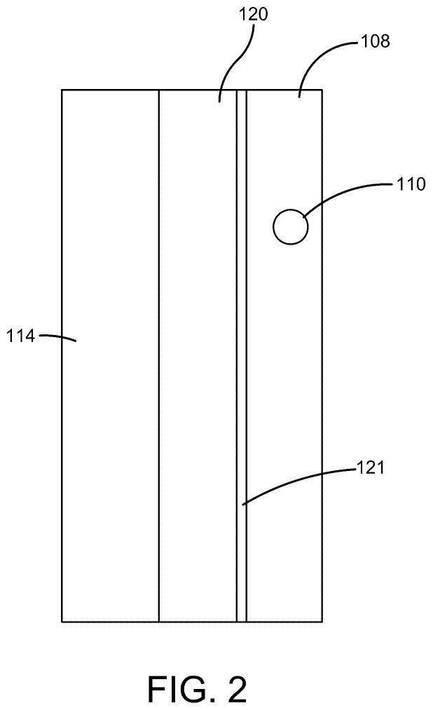

An absorbent layer 108 may be disposed within the interior of the body 102. The absorbent layer 108 may be configured to manage bodily fluids such as urine or liquid stool while resisting water saturation encountered in aquatic environments. The absorbent layer 108 may be comprised of, but is not limited to, hydrophobic nonwoven textile substrates laminated with superabsorbent polymer (SAP) elements 110, as seen in FIG. 2. The SAP elements 110 may be in the form of beads, granules, or microfibers capable of rapid fluid uptake and gel formation upon contact with excreted waste. The spatial distribution of SAP elements 110 within the absorbent layer 108 may be optimized to localize absorbency in target zones corresponding to expected waste deposition, while reducing unnecessary bulk in non-critical areas.

A containment barrier layer 120 may be positioned adjacent to or enclosing the absorbent layer 108, as seen in FIG. 2. The containment barrier layer 120 may be comprised of a flexible, stretchable, and waterproof material such as but not limited to thermoplastic polyurethane (TPU), polyurethane laminate (PUL), or polyether block amide (PEBA). The containment barrier layer 120 may serve to inhibit leakage of fluids and semi-solids from the absorbent layer 108 to the external environment. The barrier layer 120 may be configured to deform in multiple directions, accommodating movements such as flexion, extension, abduction, and rotation without rupture or displacement. The barrier layer 120 may also feature a hydrophobic finish 121 to further enhance resistance to external water infiltration during aquatic use.

Externally, the body 102 may be covered by a shell layer 114, as seen in FIG. 1. The shell layer 114 may be comprised of a swimwear-grade textile, such as but not limited to chlorine-resistant elastic polyurethane, spandex blends, or polyester-based knits. The shell layer 114 may be selected to maintain a familiar visual and tactile profile consistent with non-absorbent swimwear. The shell layer 114 may include aesthetic features or textile treatments 116, including but not limited to printed indicia in the form of patterns, jacquard weaving, embossed surface patterns, etc. These treatments 116 may serve to visually obscure the underlying absorbent and barrier components and may provide the visual appearance of conventional swimwear to ensure discretion and social acceptability during use.

In one embodiment, the device 100 may further comprise a saturation indicator strip 128, as seen in FIG. 1. The saturation indicator strip 128 may be comprised of a colorimetric material that undergoes a visible color change upon exposure to a threshold level of moisture or specific bodily waste compounds. The saturation indicator strip 128 may be a moisture-sensitive saturation indicator strip, a pH-sensitive saturation indicator strip, or other chemoresponsive saturation indicator strips that change color in response to the detection of urine and/or feces. The saturation indicator strip 128 may be embedded within or affixed to the inner surface of the absorbent layer 108 or barrier layer 120 and may be oriented to provide a clearly visible cue to a caregiver or user. The saturation indicator strip 128 may provide a non-verbal notification that the absorbent capacity of the device 100 has been reached and that disposal may be necessary.

The device 100 may further comprise tear-apart side seams 132 preferably positioned along the lateral flanks of the body 102. The tear-apart side seams 132 may be configured to rupture under moderate manual force, allowing the device 100 to be removed without requiring downward pulling over the legs. This configuration may facilitate hygienic removal of the device 100 from a user, particularly when the absorbent layer 108 is saturated or soiled. The tear-apart side seams 132 may be formed using reduced-strength bonding methods such as perforated thermal welds, ultrasonic scoring, or partial adhesive strip application. These methods may be selected to balance the mechanical integrity of the garment during normal use with ease of separation upon intentional application of force.

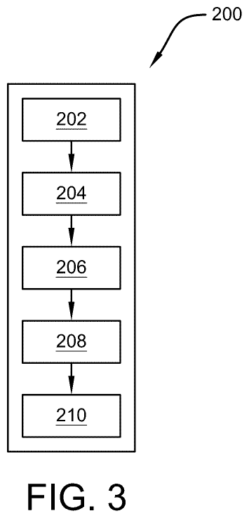

The present invention is also comprised of a method of using 200 the device 100, as seen in FIG. 3. First, a device 100 is provided comprised of a body 102 including at least one absorbent layer 108, at least one containment barrier layer 120, at least one shell layer 114, at least one saturation indicator strip 128, and tear-apart side seams 132, wherein the body 102 includes at least one waist opening 106 and at least two leg openings 104, each opening bordered with elastic bands 118 [Step 202]. Then, the device 100 can be positioned below the user's waist and pulled upward over the legs and hips until the waist opening 106 encircles the pelvic crest and the leg openings 104 align with the user's thighs, thereby securing the device 100 in place by means of the elastic bands 118 [Step 204]. Next, the user can engage in swimming or other aquatic activities while the absorbent layer 108 and containment barrier layer 120 inhibit leakage and resist saturation from environmental water [Step 206]. During or after use, the saturation indicator strip 128 can be visually inspected to determine whether the absorbent layer 108 has reached its moisture threshold, signaling the need for replacement [Step 208]. Finally, the device 100 can be removed by manually tearing the tear-apart side seams 132 and discarding the device 100 in accordance with appropriate sanitary disposal procedures [Step 210].

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “swim diaper garment device” and “device” are interchangeable and refer to the swim diaper garment device 100 of the present invention.

Notwithstanding the forgoing, the swim diaper garment device 100 of the present invention and its various components can be of any suitable size and configuration as is known in the art without affecting the overall concept of the invention, provided that they accomplish the above-stated objectives. One of ordinary skill in the art will appreciate that the size, configuration, and material of the swim diaper garment device 100 as shown in the FIGS. are for illustrative purposes only, and that many other sizes and shapes of the swim diaper garment device 100 are well within the scope of the present disclosure. Although the dimensions of the swim diaper garment device 100 are important design parameters for user convenience, the swim diaper garment device 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A swim diaper garment device comprising:
a body comprised of a waist opening and a pair of leg openings;
an absorbent layer;
a containment barrier layer positioned adjacent to or enclosing the absorbent layer;
a shell layer covering an exterior of the body; and
a tear-apart seam disposed on the body, wherein the tear-apart seam is configured to rupture under manual force.

2. The swim diaper garment device of claim 1, wherein the body is comprised of a brief, a trunk, a boxer brief, or a swim short.

3. The swim diaper garment device of claim 1 further comprised of an elastic band.

4. The swim diaper garment device of claim 1, wherein the absorbent layer is comprised of a hydrophobic nonwoven textile substrate.

5. The swim diaper garment device of claim 4, wherein the hydrophobic nonwoven textile substrate is comprised of a superabsorbent polymer element.

6. The swim diaper garment device of claim 5, wherein the superabsorbent polymer element is comprised of a bead, a granule, or a microfiber.

7. The swim diaper garment device of claim 1, wherein the containment barrier layer is comprised of a thermoplastic polyurethane, a polyurethane laminate, and a polyether block amide.

8. The swim diaper garment device of claim 1, wherein the containment barrier layer is comprised of a hydrophobic finish.

9. The swim diaper garment device of claim 1, wherein the shell layer is comprised of a chlorine-resistant elastic polyurethane, a spandex blend, or a polyester-based knit.

10. A swim diaper garment device comprising:
a body comprised of a waist opening and a pair of leg openings;
an absorbent layer;
a containment barrier layer positioned adjacent to or enclosing the absorbent layer;
a shell layer covering an exterior of the body;
a saturation indicator strip; and
a tear-apart seam disposed on the body, wherein the tear-apart seam is configured to rupture under manual force.

11. The swim diaper garment device of claim 10, wherein the saturation indicator strip is comprised of a moisture-sensitive saturation indicator strip, a pH-sensitive saturation indicator strip, or a chemoresponsive saturation indicator strip.

12. The swim diaper garment device of claim 10, wherein the body is comprised of a brief, a trunk, a boxer brief, or a swim short.

13. The swim diaper garment device of claim 10 further comprised of an elastic band.

14. The swim diaper garment device of claim 10, wherein the absorbent layer is comprised of a hydrophobic nonwoven textile substrate.

15. The swim diaper garment device of claim 14, wherein the hydrophobic nonwoven textile substrate is comprised of a superabsorbent polymer element.

16. The swim diaper garment device of claim 15, wherein the superabsorbent polymer element is comprised of a bead, a granule, or a microfiber.

17. The swim diaper garment device of claim 10, wherein the containment barrier layer is comprised of a thermoplastic polyurethane, a polyurethane laminate, and a polyether block amide.

18. The swim diaper garment device of claim 10, wherein the containment barrier layer is comprised of a hydrophobic finish.

19. The swim diaper garment device of claim 10, wherein the shell layer is comprised of a chlorine-resistant elastic polyurethane, a spandex blend, or a polyester-based knit.

20. A method of using a swim diaper garment device, the method comprising the following steps:
providing a swim diaper garment device comprised of a body comprised of an absorbent layer, a containment barrier layer, a shell layer, and a tear-apart seam, wherein the body includes a waist opening and a pair of leg openings;
positioning the swim diaper garment device below a waist of a user and pulling the swim diaper garment device upward over a leg and a hip of the user until the waist opening encircles a pelvic crest and the pair of leg openings align with a pair of thighs of the user;
engaging in aquatic activities while wearing the swim diaper garment device; and
removing the swim diaper garment device from a user by manually tearing the tear-apart seam and discarding the swim diaper garment device.
